(19) United States
(12) Patent Application Publication
(10) Pub. No.: US 2015/0045702 A1
(43) Pub. Date: Feb. 12, 2015

(54) COSMETIC DEVICE DISPOSED WITH MAGNETIC ATTRACTION STRUCTURE AND REPLACEABLE COSMETIC ASSEMBLY

(71) Applicant: MEI-CHI-NA HSINYEN CO., LTD., Taipei (TW)

(72) Inventor: Su-Lin Lin, Taipei (TW)

(21) Appl. No.: 14/454,376

(22) Filed: Aug. 7, 2014

(30) Foreign Application Priority Data
Aug. 8, 2013 (TW) 102214900

(51) Int. Cl. A61H 15/02; A61N 5/06; A61M 35/00

(52) U.S. Cl. CPC A61H 15/02; A61M 35/003; A61N 5/0616; A61N 2005/0651; A61N 2005/0659; A61N 2005/0663; A61F 2007/0052
USPC 601/19; 604/291; 607/90

(57) ABSTRACT

A cosmetic device disposed with a magnetic attraction structure and a replaceable cosmetic assembly is disclosed. The cosmetic device includes a holding unit, a connection part, a cover body, and the cosmetic assembly. The holding unit has a power supply unit and a notch disposed with a first magnetic attraction component and several first connection terminals. The power supply unit electrically connects with the first connection terminals for transmitting electric power. The connection part includes a fixing block disposed with a second magnetic attraction component and several second connection terminals. The second magnetic attraction component and the first magnetic attraction component may attract each other for binding the connection part with the holding unit. The second connection terminals are electrically connected with the first connection terminals. The cosmetic assembly assembled with the cover body, and the cosmetic assembly is electrically connected with the second connection terminals for performing cosmetic functionalities.

### RELATED APPLICATIONS

This application claims the benefit of priority to Taiwan Patent Application No. 102214900, filed on Aug. 8, 2013, which is hereby incorporated by reference in its entirety.

### BACKGROUND

1. Technical Field

The present disclosure relates to a cosmetic device disposed with a magnetic attraction structure; in particular, to a cosmetic device disposed with a magnetic attraction structure and a replaceable cosmetic assembly. Therefore, the user may be able to install the cosmetic assembly to a holding unit, for changing the cosmetic functionalities associated with different cosmetic assemblies to satisfy the actual requirements.

2. Description of Related Art

Certain people tend to be staying beautiful or maintaining the beauty. All kinds of cosmetic products for facial care and body fitness therefore are popular among beauty pursuers. Recently, almost every woman does cosmetic maintenance to her face and skins each of the days, and searches for every possible cosmetic method for achieving the goal of being beautiful or maintaining the beauty. Moreover, the skins are the facade of one's appearance, especially the fineness, shininess, and rosiness of facial skins However, while the skins may also protect human body from fierce winds and burning sunlight, they may be damaged during the process. Therefore, the maintenance of skins is an issue which should not be ignored in the cosmetic treatments. According to the improvement of the times and technologies, all kinds of newly modeled and unique functioning cosmetic devices are designed for matching the increasing market demands, such as an ultrasound cosmetic device, a negative ion cosmetic device, a laser light cosmetic device, and a far-infrared ray cosmetic device, etc., in order to provide multivariate services to the consumers, enabling the consumers to have clean, healthy, young, and even delicate skins.

That said, each conventional cosmetic device may only have one cosmetic function and is unable to provide multivariate cosmetic functionalities to the users. Additionally, since the cost and volume of the conventional cosmetic device are relatively large, that the conventional cosmetic device can only be used once gradually becomes unacceptable to the average consumers. Although some relatively costless and small portable cosmetic devices are developed, the consumers still need to buy different cosmetic devices for meeting the need of the multiple cosmetic requirements. Therefore, when the consumer is going for outdoor activities, the consumer needs to bring out several single-functioning cosmetic devices, which may occupy large volume of his or her package. In addition, the consumer needs to pay extra cost for buying the cosmetic devices which have all kinds of cosmetic functionalities. Moreover, when the consumer is using the cosmetic devices, relatively large spaces are required for the placement of the cosmetic devices, and the consumer need to frequently exchange one cosmetic device with another for achieving the multiple cosmetic functionalities. Therefore, the consumers may suffer from the un-conveniences and become more and more frustrated when using the conventional cosmetic devices.

Thus, a cosmetic device with a magnetic attraction structure and a replaceable cosmetic assembly should be developed, for saving the cost for buying extra cosmetic devices, reducing the extra space needed to put the cosmetic devices, and enabling the consumer to change different cosmetic assemblies rapidly, in order to achieve the efficacies of multivariate cosmetic functionalities.

### SUMMARY

As mentioned in above, the conventional cosmetic device may have only one cosmetic function and is unable to change the cosmetic assembly thereof to meet the different needs. For the users, the conventional cosmetic device may cause extra cost and is not convenient to carry and be used. The present disclosure has disclosed a cosmetic device with a magnetic attraction structure and a replaceable cosmetic assembly. According to the present disclosure, the user may greatly save the cost for buying extra cosmetic devices, and the storage space of the cosmetic device may be reduced, for conveniently and rapidly replacing different cosmetic assemblies in the cosmetic device, in order to realize multiple cosmetic functionalities.

One of the objectives of the present disclosure is to provide a cosmetic device with a magnetic attraction structure and a cosmetic assembly which is replaceable. The cosmetic device includes a holding unit, a connection part, a cover body, and a cosmetic assembly. A notch is concavely disposed at the top side of the holding unit, and a first magnetic attraction component and a plurality of first connection terminals are disposed in the notch. Moreover, the holding unit has a power supply unit which is electrically connected with the first connection terminals, for transmitting the electric power to the first connection terminals. A fixing block is convexly disposed at the bottom side of the connection part, and the configuration of the fixing block matches the configuration of the notch. In addition, the bottom side of the fixing block is disposed with a second magnetic attraction component and a plurality of second connection terminals. The position of the second magnetic attraction component corresponds to the position of the first magnetic attraction component, and the positions of the second connection terminals correspond to the positions of the first connection terminals respectively. When the connection part is assembled to the holding unit, the second magnetic attraction component and the first magnetic attraction component attract each other, enabling the connection part to be fixed at the holding unit. Moreover, the second connection terminals may respectively and electrically be connected with the first connection terminals, for allowing the second connection terminals to receive the electric power transmitted from the first connection terminals. The bottom side of the cover body is connected to the top side of the connection part, and the bottom side of the cover body is concavely disposed with a first containing space. The cosmetic assembly is assembled to the cover body, and the bottom side of the cosmetic assembly is electrically connected with the second connection terminals, for the cosmetic assembly to be associated with the multiple cosmetic functionalities.

Therefore, when the user is using the cosmetic device shown in the present disclosure for cosmetic maintenance to the user's face or body, he or she may be able to rapidly replace the different cosmetic assemblies which have different cosmetic functionalities to meet the actual requirements. The cosmetic assemblies may roll or slide along the surface of the face or the body of the user, for providing the face or body muscles with the stress relaxing, smoothing the nerves of the face or body of the user, and whitening the skins of the face or body of the user. In addition, due to the magnetic attraction structure of the cosmetic device, the user may easily install the cosmetic assembly to the holding unit, or remove the cosmetic assembly from the holding unit, so the user may be able to exchange the cosmetic assemblies to meet the need of the different requirements. When the user is going outdoors, only a single holding unit needs to be carried around collocating with different cosmetic assemblies, and the requirements of different cosmetic functionalities can easily be met. Therefore, the cosmetic device is convenient to be used and carried.

For further understanding of the present disclosure, reference is made to the following detailed description illustrating the embodiments and examples of the present disclosure. The description is only for illustrating the present disclosure, not for limiting the scope of the claim.

### BRIEF DESCRIPTION OF THE DRAWINGS

The drawings included herein provide further understanding of the present disclosure. A brief introduction of the drawings is as follows:

FIG. 1 shows a three dimensional schematic diagram of a connection device according to a first embodiment of the present disclosure;

FIG. 2 shows a three dimensional schematic diagram of a connection device according to a first embodiment of the present disclosure;

FIG. 3 shows a three dimensional schematic diagram of a connection device according to a second embodiment of the present disclosure; and

FIG. 4 shows a three dimensional schematic diagram of a connection device according to a third embodiment of the present disclosure.

### DESCRIPTION OF THE EXEMPLARY EMBODIMENTS

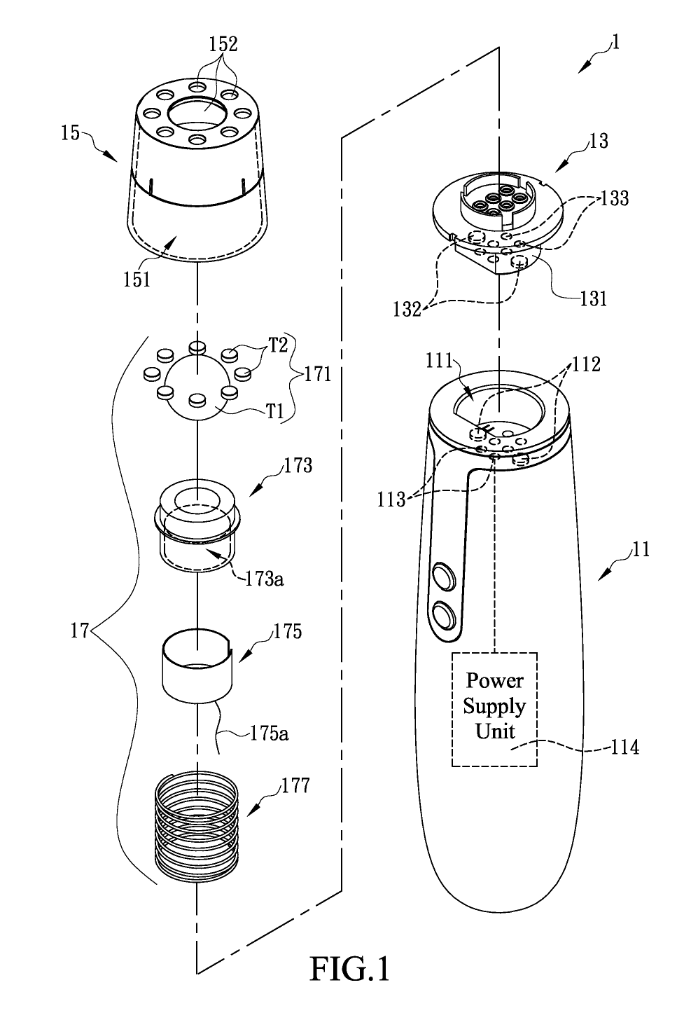

The present disclosure is a cosmetic device 1 with a magnetic attraction structure and a replaceable cosmetic assembly. Please refer to FIG. 1 which shows a first embodiment according to the present disclosure. The cosmetic device 1 includes a holding unit 11, a connection part 13, a cover body 15, and a cosmetic assembly 17. The top side of the holding unit 11 is disposed with a notch 111. A first magnetic attraction component 112 (such as a magnet or a magnetic permeability piece) and a plurality of first connection terminals 113 are disposed in the notch 111. In addition, the holding unit 11 has a power supply unit 114 which is electrically connected with the first connection terminals 113, for transmitting electric power to the first connection terminals 113. A fixing block 131 is convexly disposed at the bottom side of the connection part 13. The configuration of the fixing block 131 matches the configuration of the notch 111, and a second magnetic attraction component 132 (such as a magnet or a magnetic permeability piece) and a plurality of second connection terminals 133 are disposed on the bottom side of the fixing block 131. The position of the second magnetic attraction component 132 corresponds to the position of the first magnetic attraction component 112, and the positions of the second connection terminals 133 correspond to the positions of the first connection terminals 113. When the connection part 13 is assembled or installed to the holding unit 11, the second magnetic attraction component 132 and the first magnetic attraction component 112 can attract each other, enabling the connection part 13 to be fixed at the holding unit 11. Moreover, the second connection terminals 133 may be electrically and respectively connected with the first connection terminals 113, for allowing the second connection terminals 133 to receive the electric power transmitted from the first connection terminals 113. A first containing space 151 is concavely disposed in the bottom side of the cover body 15. A plurality of openings 152 meanwhile are disposed at the top side of the cover body 15. The openings 152 are connected with the first containing space 151. The cosmetic assembly 17 is installed or assembled to the cover body 15, and the bottom side of the cosmetic assembly 17 is electrically connected with the second connection terminals 133. With the above arrangement, the cosmetic assembly 17 is able to perform cosmetic functionalities.

Please refer to FIG. 1 again. In the first embodiment, the cosmetic assembly 17 includes a cosmetic component 171, a bearing base 173, and an electro-thermal piece 175. The cosmetic component 171 is installed in the first containing space 151. The bearing base 173 is also disposed in the first containing space 151, and a second containing space 173a is concavely disposed at the bottom side of the bearing base 173. The top side of the bearing base 173 is abutted on the bottom side of the cosmetic component 171. The electro-thermal piece 175 is disposed in the second containing space 173a and is abutted on the inner wall surface of the bearing base 173. The electro-thermal piece 175 includes a wire 175a, for receiving the electric power transmitted from the second connection terminals 133. After the electric power is converted to heat, such heat may be transmitted to the bearing base 173, allowing the cosmetic component 171 to perform cosmetic functionalities after the cosmetic component 171 receives the heat from the bearing base 173.

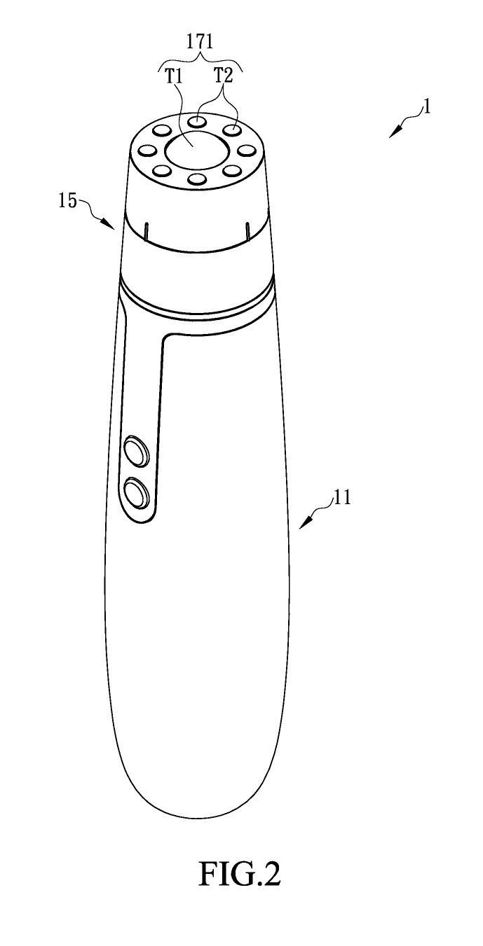

Please refer FIGS. 1 and 2. In the first embodiment of the present disclosure, the cosmetic component 171 includes at least one ceramic bead T1 and a plurality of germanium stones T2. Each of the top sides of the ceramic beads T1 and the germanium stones T2 is exposed out of the top side of the cover body 15, and each of the bottom sides of the ceramic beads T1 and the germanium stones T2 is abutted on the top side of the bearing base 173. Thus, the ceramic bead T1 may be able to emit far-infrared rays after the ceramic bead T1 receives the heat transmitted from the bearing base 173, and the germanium stones T2 may be able to release negative ions after the germanium stones T2 receive the heat. When the user uses the ceramic bead T1 and the germanium stones T2 to roll on the face, shoulder, or neck, the cosmetic efficacies including reducing muscle stresses, smoothing nerves, warming emotions, recovering energy, and whitening skins may be achieved.

The far-infrared rays are parts of the sun light, and may be in form of the electromagnetic waves. The wavelength of the far-infrared ray ranges from about 4 micrometers to 400 micrometers, and associates with the low energy which is unable to hurt the human body. The far-infrared ray is not the ionizing radiation. In addition, the human body is the organism which also emits far-infrared rays having about 9 micrometers wavelength. Moreover, the far-infrared ray is the part of the sun light which can penetrate into the deepest skins and subcutaneous tissues, and can promote the blood circulation for keeping the human body warm, energetic, and healthy. Therefore, the Japanese people call the far-infrared rays as “fertility lights” and claim that the far-infrared rays include the following three major features:

1. Radiation: The far-infrared rays have the same activity status as the lights, and can transmit to the objects without the airs. For example, the sun lights are able to transmit through the vacuum in the universe for reaching the Earth, and that is the radiation property of the far-infrared rays.

2. Penetration capability: The differences between the far-infrared rays and the visible lights include that the far-infrared rays have strong penetration capability. The far-infrared rays are able to penetrate the human body about 4 centimeters to 10 centimeters, which can go deep into the subcutaneous tissues to warm up the body from the inside and to provide the with the cells.

3. Resonance absorption: Every substance is formed by numerous of molecules, and the molecule is formed by atoms (or atomic groups). Thus, the substances may resonate through certain vibrations. The vibration number of the far-infrared rays and the vibration number of the biological molecules are almost in the same range. When the far-infrared rays reach the substances, the resonance absorption phenomenon is generated in the molecules of the substances, for increasing the activation energy of the molecules. Therefore, the far-infrared rays not only warm up the body but also activate the body functionalities.

The element germanium is 14(IV) group element which is formed by 32 electrons, and the atomic number thereof is 32 and the atomic mass thereof is 72.64 g/mol. The germanium is the semiconductor metal between the metal and non-metal elements, and has great ionic permeability. In addition, the germanium may release negative ions when the temperature reaches 32 degrees Celsius, and the negative ions may be able to increase the body temperature, improve the blood circulation, and ease the muscle discomfort and metabolic fatigues. Moreover, the negative ions can also absorb the hydrogen ions with positive electricity, and can neutralize the acid and help the body to restore its normal functionalities. For example, the negative ions can absorb the positive ions, such as the hydrogen ions in the blood, for restoring the blood with weak acid to the normal status. Additionally, the negative ions can also neutralize the ions in the human body for adjusting biological currents, in order to maintain the status of the body in balance.

Please refer to FIG. 1. In a first embodiment according to the present disclosure, the cosmetic assembly 17 further includes a spring 177 which is disposed between the bearing base 173 and the connection part 13, for allowing the bearing base 173 and the ceramic bead T1 to be driven by the elastic restoring force of the spring 177. Therefore, when the user rolls the ceramic bead T1 on the face or different body parts thereof, the ceramic bead T1 is able to stick close to and roll along the body curves of the user because of the elastic restoring force of the spring 177, in order to apply relatively better cosmetic functionalities to the skins of the user.

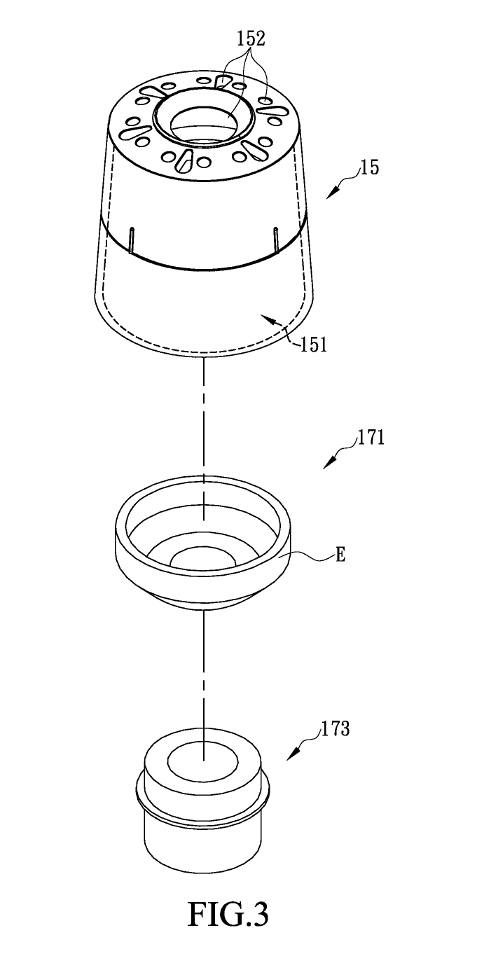

In the second embodiment according to the present disclosure, please refer to FIG. 3, the holding unit, the connection part, and the cover body therein are the same as their counterparts in the aforementioned embodiment, which are not repeatedly described. Please refer to FIGS. 1 and 3 again, the cosmetic component 171 in the second embodiment is an essential oil bearing component E (for example, a plate-shaped object which is made by porous materials and is able to absorb the essential oil, or a plate-shaped object which is able to bear the essential oil). The essential oil bearing component E is disposed in the first containing space 151, for accommodating the essential oil. In addition, the bottom side of the essential oil bearing component E is abutted to the top side of the bearing base 173. When the user puts the essential oil into the essential oil bearing component E for receiving the heat transmitted from the bearing base 173, the essential oil may evaporate and be exhausted out of the cover body 15 through the openings 152, for allowing the user to smell the essential oil in order to achieve the efficacies of cleaning and smoothing the spirit, anger, and fatigue. The essential oil commonly exists in each part of the plants including the tree root, the tree trunk, the tree bark, the tree stem, the leaf, and the flower. In fact, the essential oil is not oil, it is actually an organic compound generated through photosynthesis, and is aromatic plant group with complex content having the functionalities for smoothing the spirit and easing the fatigue.

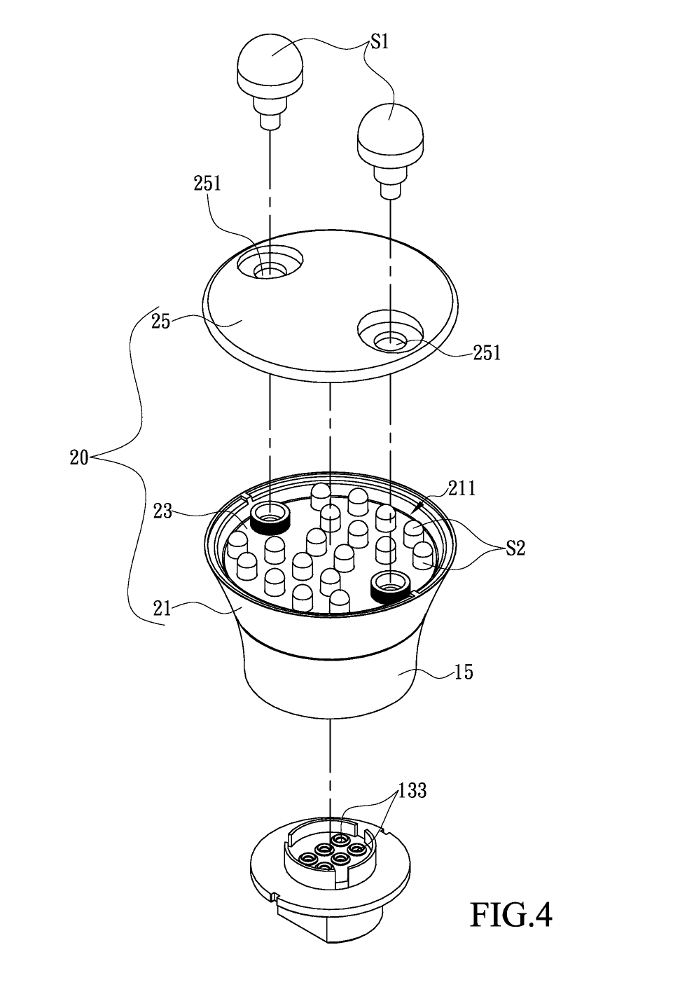

Please refer to FIG. 4 which shows a cosmetic device according to a third embodiment of the present disclosure, and the structures of the holding unit, the connection part, and the cover body are the same as their counterparts in the mentioned embodiments, which are not repeatedly described. Please refer to FIGS. 1 and 4 again. The cosmetic assembly 20 of the third embodiment includes a fixing base 21, a circuit board 23, and a microwave translucent mask 25. The bottom side of the fixing base 21 is abutted to the top side of the cover body 15, and a third containing space 211 is concavely disposed at the top side of the fixing base 21. The circuit board 23 is disposed in the third containing space 211, and is able to receive electric power transmitted from the second connection terminals 133 through a wire. There are several guided wave components S1 and red light diodes S2 disposed on the circuit board 23, and the guided wave components S1 and the red light diodes S2 are able to be electrically connected with the second connection terminals 133 through the circuit board 23. The microwave translucent mask 25 is disposed at the top side of the fixing base 21, for enabling the circuit board 23, the guided wave components S1, and the red light diodes S2 to be accommodated within the third containing space 211. In addition, the guided wave components S1 may generate electro magnetic susceptibility (EMS) after receiving the electric power. The EMS imports trace current 1800 to 3600 times per minute. After the current is used on the face along with cosmetic cream, it may be able to tighten the facial curves, dilute the facial lines, tighten and firming the skins, and sculpture the facial curves. In addition, the current and the cream may also activate the dermis to proliferate collagen for recovering the elastic fibers of the skins

The red light diodes S2 are the so called LED (light emitting diode), and the red lights generated thereby can achieve the functionalities of activating the facial cells of the user and increasing the proliferation of the collagen. Therefore, in the present days, the LED is used not only in illuminating devices but also in medical cosmetology field. The professional medical cosmetology doctor Jin-Yuan Zhong indicates it is proven by American researches that the usage of the red lights generated by the LED is able to improve the growth of the skin cells, and the growth rate thereof are five times quicker than the normal value. Moreover, the LED phototherapy rejuvenation is the newest and the most out-breaking maintaining technique, which uses the LED lighting source for stimulating the fiber mother cells of the dermis, in order to proliferate the collagens, to increase the metabolism of the aging cells, and to rejuvenate the skin. According to clinical trials, the cosmetic functionality of improving pigment spots is optimal after going through several times of illuminations, the blood circulation of the skin may be improved, the proliferation of the collagens may be improved by stimulating the fiber mother cells of the dermis, and the functionality of the metabolism of the aging cells may be rediscovered.

Therefore, the user is able to select from the several cosmetic assemblies with different cosmetic functions when using the cosmetic device in the present disclosure, for rolling or sliding over the face or other parts of the body of the user, in order to achieve the cosmetic functionalities including releasing muscular pressures, smoothing nerves, and whitening skins. In addition, because the cosmetic device has a magnetic attraction structure, the user may be able to easily and rapidly install or uninstall all kinds of cosmetic assemblies, for changing different cosmetic assemblies with different cosmetic functionalities according to the actual requirements. Moreover, when the user goes for outdoor activities, he or she only needs to carry one holding unit along with different cosmetic assemblies, for implementing different cosmetic functionalities, which is very convenient when it comes to device carrying and using.

Some modifications of these examples, as well as other possibilities will, on reading or having read this description, or having comprehended these examples, will occur to those skilled in the art. Such modifications and variations are comprehended within this disclosure as described here and claimed below. The description above illustrates only a relative few specific embodiments and examples of the present disclosure. The present disclosure, indeed, does include various modifications and variations made to the structures and operations described herein, which still fall within the scope of the present disclosure as defined in the following claims.

What is claimed is:

1. A cosmetic device disposed with a magnetic attraction structure and a replaceable cosmetic assembly, comprising:
a holding unit, wherein a notch is concavely disposed at a top side of the holding unit, a first magnetic attraction component and a plurality of first connection terminals are disposed in the notch, a power supply unit is disposed in the holding unit, and the power supply unit is electrically connected with the first connection terminals for transmitting electric power to the first connection terminals;
a connection part, wherein a fixing block is convexly disposed at a bottom side of the connection part, a configuration of the fixing block matches with a configuration of the notch, a second magnetic attraction component and a plurality of second connection terminals are disposed at a bottom side of the fixing block, the position of the second magnetic attraction component corresponds to the position of the first magnetic attraction, and the positions of the second connection terminals correspond to the positions of the first connection terminals, when the connection part is assembled with the holding unit, the second magnetic attraction component and the first magnetic attraction component attract each other for enabling the connection part to be fixed at the holding unit, and the second connection terminals are electrically connected with the first connection terminals for allowing the second connection terminals to receive the electric power transmitted from the first connection terminals;
a cover body including a bottom side which is connected with a top side of the connection part, wherein a first containing space is concavely disposed in the bottom side of the cover body; and
the cosmetic assembly assembled with the cover body, wherein a bottom side of the cosmetic assembly is electrically connected with the second connection terminals, for allowing the cosmetic assembly to perform cosmetic functionalities accordingly.

2. The cosmetic device according to claim 1, wherein the cosmetic assembly includes:
a cosmetic component disposed in the first containing space;
a bearing base disposed in the first containing space, wherein a second containing space is concavely defined in a bottom side of the bearing base, and a top side of the bearing base abuts on a bottom side of the cosmetic component; and
an electro-thermal piece disposed in the second containing space and abutting on an inner wall surface of the bearing base, wherein the electro-thermal piece is disposed with a wire for receiving the electric power transmitted from the second connection terminals, and after the electric power is converted into heat, the heat is transmitted to the bearing base by the electro-thermal piece for allowing the cosmetic component to perform the cosmetic functionalities after the cosmetic component receives the heat transmitted from the bearing base.

3. The cosmetic device according to claim 2, wherein a top side of the cover body is disposed with a plurality of openings, and the openings communicate with the first containing space.

4. The cosmetic device according to claim 3, wherein the cosmetic component is an essential oil bearing component disposed in the first containing space for accommodating essential oil, and a bottom side of the essential oil bearing component is abutted on the top side of the bearing base, for allowing the essential oil in the essential oil bearing component to evaporate and be transpired through the openings after the essential oil bearing component receives the heat transmitted from the bearing base.

5. The cosmetic device according to claim 4, wherein the essential oil bearing component is made by porous materials, and is shaped as a plate capable of absorbing the essential oil.

6. The cosmetic device according to claim 4, wherein the essential oil bearing component is shaped as a plate capable of bearing the essential oil.

7. The cosmetic device according to claim 3, wherein the cosmetic component includes at least one ceramic bead and a plurality of germanium stones, each of the top sides of the ceramic beads and the germanium stones respectively passes through the corresponding opening and is exposed out of the top side of the cover body, a bottom side of the ceramic bead is abutted on the top side of the bearing base for allowing the ceramic bead to emit far-infrared ray after the ceramic bead receives the heat transmitted from the bearing base, and the germanium stones emit negative ions after the germanium stones receive the heat.

8. The cosmetic device according to claim 7, wherein the cosmetic assembly further includes a spring disposed between the bearing base and the connection part, for allowing the bearing base and the ceramic bead to be driven by the elastic restoring force of the spring.

9. The cosmetic device according to claim 1, wherein the cosmetic assembly includes:
a fixing base, wherein a bottom side of the fixing base is disposed to a top side of the cover body, and a third containing space is concavely disposed at a top side of the fixing base;
a circuit board disposed in the third containing space, wherein the circuit board receives the electric power transmitted from the second connection terminals through a wire, a plurality of guided wave components and a plurality of red light diodes are disposed on the circuit board, and the guided wave components and the red light diodes are electrically and respectively connected with the second connection terminals through the circuit board; and
a microwave translucent mask disposed at the top side of the fixing base, for allowing the circuit board, the guided wave components, and the red light diodes to be accommodated in the third containing space.

10. The cosmetic device according to claim 9, wherein a top side of the microwave translucent mask is disposed with a plurality of openings, and the openings communicate with the third containing space.

11. The cosmetic device according to claim 10, wherein the guided wave components are disposed on the circuit board respectively through the openings, and the guided wave components are exposed out of the top side of the microwave translucent mask.
